(19) United States
(12) Patent Application Publication
(10) Pub. No.: US 2026/0286351 A1
(43) Pub. Date: Sep. 24, 2026

(54) METHOD FOR CONTROLLING INTRACELLULAR LOCALIZATION OF SGRNA AND REGULATING GENE EXPRESSION

(71) Applicant: Guangzhou Medical University, Guangzhou (CN)

(72) Inventors: Xi-Yong Yu, Guangzhou (CN); Jianfeng Qin, Guangzhou (CN); Ao Shen, Guangzhou (CN); Yuguang Zhu, Guangzhou (CN); Jishuo Chang, Guangzhou (CN)

(21) Appl. No.: 19/460,349

(22) Filed: Jan. 27, 2026

(30) Foreign Application Priority Data
Mar. 20, 2025 (CN) 202510335053.7

(51) Int. Cl. C12N 15/11; C07K 14/005; C07K 14/47; C12N 9/22; C12N 15/85

(52) U.S. Cl. CPC C12N 15/111; C07K 14/005; C07K 14/4702; C12N 9/226; C12N 15/85; C07K 2319/095; C12N 2310/20; C12N 2795/18022; C12N 2830/002

(57) ABSTRACT

A light-controlled element NES-Ms2-Lans5 includes, sequentially in an order from 5′ to 3′, a NES gene fragment, an Ms2 gene fragment, and a Lans5 gene fragment. Each gene fragment is linked via a linker. A light-controlled CRISPR component including the aforementioned light-controlled element, a gene targeting element, and a gene regulatory element is provided. According to the light-controlled CRISPR component, a method for light-controlled target gene expression is provided. A strategy to achieve gene editing by regulating an intracellular localization of sgRNA is firstly provided, enabling activation and expression of specific genes.

### CROSS-REFERENCE TO RELATED APPLICATION

This application claims priority to Chinese Patent Application No. 202510335053.7, filed on Mar. 20, 2025, which is herein incorporated by reference in its entirety.

### TECHNICAL FIELD

The disclosure relates to the field of biotechnologies, and more particularly to a light-controlled element, a light-controlled clustered regularly interspaced short palindromic repeats (CRISPR) component, and a method for controlling an intracellular localization of a single guide ribonucleic acid (sgRNA) to regulate gene expression.

### STATEMENT REGARDING SEQUENCE LISTING

The sequence listing associated with this application is provided in text format in lieu of a paper copy and is hereby incorporated by reference into the specification. The name of the XML file containing the sequence listing is 25089THXT-USP1-SL. The XML file is 30124 bytes; is created on Jan. 23, 2026; and is being submitted electronically via patent center.

### BACKGROUND

CRISPR/CRISPR-associated protein 9 (Cas9) is a gene editing technology that utilizes sgRNA to guide a Cas9 enzyme to precisely recognize and cleave target DNA sequences. Dead Cas9 (dCas9), as a variant of Cas9, is generated by mutating nuclease domains of Cas9, resulting in loss of deoxyribonucleic acid (DNA)-cutting activity while retaining an ability to bind to DNA sequences. In this way, by fusion-expressing different transcriptional activators or repressors, the dCas9 can precisely control gene expression. In this process, sgRNA serves as a critical component of a CRISPR/dCas9 system, capable of guiding a dCas9 enzyme to recognize specific DNA sequences. The Cas9 or the dCas9 must form a complex with the sgRNA within cell nucleus to exert functions in gene editing or gene regulation.

Optogenetics is a technology that uses light to control cellular fate, integrating optical and genetic techniques. The optogenetics targets suitable exogenous light-sensitive proteins (photosensitive proteins) into specific living cells via genetic methods, then stimulates the photosensitive proteins by light with specific wavelengths, thereby controlling cellular activities and even animal behaviors. Specifically, the photosensitive proteins are key to achieving the optogenetics. Currently, commonly used photosensitive proteins include cryptochrome 2 (CRY2), bacterial phytochrome sensor (BphS), phototropin 1 light-oxygen-voltage 2 (LOV2) domain, etc. These photosensitive proteins, upon illumination of light with specific wavelengths, undergo binding or dissociating with other proteins, or structural changes, thereby achieving various functions. In addition, a photosensitive element Lans4 protein, engineered based on an LOV2 protein, can precisely regulate its intracellular position through light illumination. This protein has nuclear export signals (NES) and nuclear localization signals (NLS) at both terminals, respectively. Among them, the NLS at a C-terminal exhibits unique light-responsive characteristics. Under dark conditions, this NLS is hidden due to protein folding and thus unable to function. When stimulated by light, the LOV2 protein undergoes conformational changes, leading to exposure of the NLS at the C-terminal, thereby activating functions of the NLS to drive fusion proteins to enter the cell nucleus.

Currently, there are mainly two types of light-controlled gene editing technologies that combine the optogenetics and gene editing techniques. One type is a fluorescence-activating and absorption-shifting tag (FAST) system, which consists of two components: N-terminal Cas9 fused with cohesin domain 2 (Coh2) protein (Cas9(N)-Coh2) and C-terminal Cas9 fused with dockerin domain S (DocS) protein (DocS-Cas9(C)). Under far-red light illumination, the N-terminal Cas9 and C-terminal Cas9 interact through Coh2 and DocS and bind together to form a complete Cas9 nuclease, thereby exerting its gene editing function. Another type is a near-infrared light-controlled gene editing system, which uses gold nanorods as carriers to load CRISPR plasmids. Under illumination, the gold nanorods convert light energy into heat energy, activating a thermosensitive promoter to drive Cas9 expression, thereby enabling gene editing.

The light-controlled gene editing technologies in the art primarily rely on altering a structure and activity of the Cas9 or the dCas9 through light signals. However, a strategy to achieve gene editing by regulating an intracellular localization of sgRNA has not been reported yet.

### SUMMARY

The disclosure aims to provide a light-controlled element NES-MS2 bacteriophage coat protein (Ms2)-light-activated nuclear shuttling (Lans5), a light-controlled CRISPR component, and a method for controlling intracellular localization of sgRNA to regulate gene expression.

The aforementioned objectives are achieved through the following technical solutions.

In a first aspect, the disclosure provides the light-controlled element NES-Ms2-Lans5, including, sequentially in an order from 5′ to 3′, a NES gene fragment, an Ms2 gene fragment, and a Lans5 gene fragment. Each gene fragment is connected via a linker.

In some embodiments, the NES gene fragment is as shown in SEQ ID NO: 1.

In some embodiments, the Ms2 gene fragment is as shown in SEQ ID NO: 2.

In some embodiments, the Lans5 gene fragment is as shown in SEQ ID NO: 3.

In some embodiments, the linker is the amino acid sequence of GSGSGSGSG as shown in SEQ ID NO: 11 or the amino acid sequence of GGGGSGGGGS as shown in SEQ ID NO: 12.

In a second aspect, the disclosure provides a vector expressing the light-controlled element NES-Ms2-Lans5 as described above.

In some embodiments, the vector is a plasmid.

In a third aspect, the disclosure provides a light-controlled CRISPR component, and the light-controlled CRISPR component consists of three plasmids expressing specific proteins and ribonucleic acids (RNA).

The light-controlled CRISPR component includes the vector expressing the light-controlled element NES-Ms2-Lans5 as described above, a vector expressing an sgRNA-modified gene targeting element (also referred to as sgRNA-modified plasmid), and a vector expressing a gene regulatory element dCas9-heat shock factor 1 (hsf1)-virus protein r (vpr) (also referred to as dCas9-hsf1-vpr plasmid);
the vector expressing the gene regulatory element dCas9-hsf1-vpr is a plasmid obtained by inserting an hsf1 gene fragment into a dCas9-vpr plasmid; and
the sgRNA-modified plasmid includes an sgRNA target sequence specific to a target gene, an Ms2-binding-sequence capable of specifically binding to the Ms2 gene fragment, and enzyme cleavage sites at both ends of the sgRNA target sequence.

In some embodiments, the vector refers to biological materials from which proteins and RNAs of relevant elements can be obtained, such as various expression plasmids.

In some embodiments, the Ms2-binding-sequence is ACATGAGGATCACCCATGT, as shown in SEQ ID NO: 13.

In some embodiments, BsmBI enzyme cleavage sites are located at both ends of the sgRNA target sequence of the sgRNA-modified plasmid.

In some embodiments, the sgRNA-modified gene targeting element includes the sequence as shown in SEQ ID NO: 8.

In some embodiments, the hsf1 gene fragment is as shown in SEQ ID NO: 7.

In some embodiments, the dCas9-hsf1-vpr plasmid is a plasmid obtained by inserting the hsf1 gene fragment into a dCas9-vpr plasmid.

In some embodiments, construction steps for the dCas9-hsf1-vpr plasmid includes: linearizing the dCas9-vpr plasmid using single-enzyme digestion with MluI to obtain linearized dCas9-vpr vector, performing a homologous recombination reaction on the linearized dCas9-vpr vector with the hsf1 gene fragment bearing homology arms at 50 degrees Celsius (° C.) for 30 minutes (min) to obtain a recombination product, in which a molar ratio of the hsf1 gene fragment to the linearized dCas9-vpr vector is 3:1, with a total mass of 50 nanograms (ng) and a total volume of 10 microliters (μL); immediately transforming the recombination product into competent cells, followed by culturing to obtain the dCas9-hsf1-vpr plasmid.

In a fourth aspect, the disclosure provides an application method of the light-controlled CRISPR component as described above, including performing light-controlled gene expression using the light-controlled CRISPR component and applying the light-controlled CRISPR component as described above for preparing a kit for the light-controlled gene expression.

In a fifth aspect, the disclosure provides a method for light-controlled target gene expression, including the following steps:
S1, acquiring the light-controlled CRISPR component, in which the light-controlled CRISPR component includes the vector expressing the light-controlled element NES-Ms2-Lans5, the vector expressing the sgRNA-modified gene targeting element, and the gene regulatory element dCas9-hsf1-vpr;
S2, co-transfecting the vector expressing the light-controlled element NES-Ms2-Lans5 and the gene regulatory element dCas9-hsf1-vpr plasmid in cells containing the target gene;
S3, after co-transfecting the vector expressing the light-controlled element NES-Ms2-Lans5 and the gene regulatory element dCas9-hsf1-vpr plasmid for 24 hours (h), transfecting the vector expressing the sgRNA-modified gene targeting element; and
S4, performing illumination according to needs of target gene expression.

In some embodiments, a light for the illumination is blue light with a wavelength in a range of 460 nanometers (nm) to 470 nm, and the illumination applies in cycles of 9 min to 11 min of light followed by 2.5 min to 3.5 min of dark, for a total duration of 5.5 h to 6.5 h.

The disclosure provides a method for achieving gene regulation by altering an intracellular localization of sgRNA via light signals and designs suitable and specifically composed light-controlled element NES-Ms2-Lans5, the vector for correspondingly expressing the sgRNA-modified gene targeting element (the sgRNA-modified plasmid), and the dCas9-hsf1-vpr plasmid. In this regulatory method, first, a specific Ms2-binding-sequence is introduced into the vector of the sgRNA to obtain sgRNA-modified, thereby achieving specific recognition and binding between the sgRNA-modified and Ms2 protein in the light-controlled element NES-Ms2-Lans5. Then, by fusion expression of the Ms2 protein and Lans5 protein, a purpose of regulating intracellular localizations of the Ms2 protein and the sgRNA-modified bound to the Ms2 protein using the light signals is achieved. Only when the sgRNA-modified is controllably transported into cell nucleus and forms a complex with a dCas9 protein can a gene regulation function be realized. This invention provides a first strategy to achieve gene editing by regulating the intracellular location of the sgRNA.

The disclosure has the following beneficial effects.
1. Innovative sgRNA strategy: the disclosure proposes a novel light-controlled gene editing strategy that achieves light-controlled gene editing through the sgRNA. This differs from light-controlled systems in the art that rely on structural changes of Cas9 or dCas9 proteins, providing a novel means for gene regulation.
2. Higher spatiotemporal resolution and flexibility: the disclosure utilizes the light signals to control whether the sgRNA functions within cells. The system provided by the disclosure achieves precise activation of gene expression when needed, thereby offering higher spatiotemporal resolution and flexibility.
3. Multiplex gene editing capability: the system provided by the disclosure can design multiple sgRNAs (sgRNA target sequences for different target genes are designed and replaced into sgRNA-modified vectors) to simultaneously target multiple gene loci, providing a new tool for treatment of multigene diseases.
4. Reversibility: light-controlled activation of the sgRNA provides reversibility and dynamic regulation capabilities for gene expression.

To sum up, the disclosure provides a new, more precise, and more efficient solution for light-controlled gene editing technology by introducing the sgRNA strategy, which has great potential for application in multiple fields.

### BRIEF DESCRIPTION OF DRAWINGS

FIG. 1A through FIG. 1B illustrate schematic diagrams of a Lans4-mCherry plasmid, where FIG. 1A illustrates a schematic diagram of a linearized vector, and FIG. 1B illustrates a plasmid map.

FIG. 2 illustrates a schematic diagram of experimental results of intracellular localization and movement of the Lans4-mCherry plasmid under illumination according to embodiment 1 of the disclosure.

FIG. 3A through FIG. 3C illustrate schematic diagrams of a light-controlled element NES-Ms2-Lans5-mCherry plasmid according to embodiment 2 of the disclosure, where FIG. 3A illustrates a schematic diagram of a linearized vector, FIG. 3B illustrates a schematic diagram of linearized vectors and light-controlled results for plasmids N-1 to N-4, and FIG. 3C illustrates a map of the NES-Ms2-Lans5-mCherry plasmid.

FIG. 4 illustrates a schematic diagram of experimental results of intracellular localization and movement of the light-controlled element NES-Ms2-Lans5-mCherry plasmid under illumination according to the embodiment 2 of the disclosure.

FIG. 5A through FIG. 5E illustrates schematic diagrams of an sgRNA-modified plasmid, where FIG. 5A illustrates a schematic diagram of a linearized vector, FIG. 5B illustrates an expected sequence (only showing a part of the sequence as shown in SEQ ID NO: 16), FIG. 5C illustrates a plasmid map, FIG. 5D illustrates a sequencing result (only showing a part of the sequence as shown in SEQ ID NO: 17), and FIG. 5E illustrates an activation test result of commercially purchased dCas9-vpr.

FIG. 6 illustrates a schematic result diagram of light activation experiments targeting a human NK2 Homeobox 5 (NKX2-5) gene according to embodiment 4 of the disclosure.

FIG. 7 illustrates a schematic result diagram of light activation experiments targeting a human glyceraldehyde-phosphate dehydrogenase (GAPDH) gene according to the embodiment 4 of the disclosure.

### DETAILED DESCRIPTION OF EMBODIMENTS

In order to facilitate understanding of the disclosure, the disclosure will be described more comprehensively below. The disclosure can be implemented in various different forms and is not limited to embodiments described herein. On the contrary, these embodiments are provided to make the understanding of the disclosure more thorough and comprehensive.

Experimental methods without specific conditions specified in the following embodiments are usually in accordance with conventional conditions or conditions suggested by manufacturer. Various common chemical reagents used in the embodiments are all commercially available products.

Unless otherwise defined, all technical and scientific terms used in the disclosure have the same meaning as commonly understood by those skilled in the art of the disclosure. The terms used in the specification of the disclosure are only for a purpose of describing specific embodiments and are not used to limit the disclosure. A term “and/or” as used in the disclosure includes any and all combinations of one or more related listed items.

A linker, also known as a linker peptide, can be a common linker sequence used to connect polypeptides. The linker can connect two polypeptides and naturally fold them into a desired structure. Typically, the linker is a short peptide with hydrophobicity and a certain degree of stretchability. The linker peptide can be flexible, and in some embodiments, a flexible linker peptide is advantageous, as it can connect two proteins while maintaining their respective activity and function, such as GSGSGSGSG as shown in SEQ ID NO: 11 or GGGGSGGGGS as shown in SEQ ID NO: 12.

Strategy of Controlling an Intracellular Position of a Core Protein dCas9 Through Illumination

During initial construction, it is found that when Lans4, dCas9, hsf1, vpr, and mCherry are directly constructed into a single plasmid to form a fusion protein with a Lans4-dCas9-hsf1-vpr-mCherry structure, the fusion protein fails to achieve effective shuttling in and out of cell nucleus. This raised questions regarding whether the Lans4 is influenced by other fusion proteins, leading to speculations about issues including spatial hindrance or conformational interference.

Flexible linkers are attempted to be added: to address this issue, flexible linker peptides (such as GSGSGS as shown in SEQ ID NO: 14) are added among different elements. In theory, the flexible linkers should not affect functions of upstream and downstream proteins, as they can alleviate the spatial hindrance to some extent and provide more flexible space for protein-protein interactions. However, experimental results indicate that simply adding linkers cannot solve the issue, suggesting that inability to achieve regulation may not only be due to the spatial hindrance, but may also involve other factors such as protein-protein interaction patterns and expression levels.

Subsequently, a position of the Lans4 is considered to be adjusted: considering that structures of other fusion proteins before and after the Lans4 may influence conformational changes of the Lans4, thereby affecting functional performance of the Lans4, the position of the Lans4 is adjusted. Multiple attempts are made in total, involving altering a relative position of the Lans4 within the single plasmid (i.e., Lans4-dCas9-hsf1-vpr-mCherry) to observe whether it could improve performance of the single plasmid. However, no definitive positive results are ultimately obtained. This suggests that the issue may be more complex, involving a combined effect of multiple factors.

Strategy of Controlling an Intercellular Position of sgRNA Through Light Illumination

Optimization strategy based on nuclear pore limitation: a size of a nuclear pore of the cell nucleus is limited to a certain extent, with a pore size of about 40 nm to 60 nm, and fusion proteins that are too large in size may result in their inability to effectively pass through the nuclear pore to enter the cell nucleus. Sizes of the fusion proteins may be a reason why they fail to effectively penetrate the nuclear pore to enter the cell nucleus.

Ms2 protein is a bacteriophage protein capable of recognizing and binding to specific RNA sequences. Based on this, the disclosure proposes an innovative strategy to control the sgRNA using the Ms2 protein and an sgRNA containing an Ms2-binding-sequence. Specifically, the disclosure designs an sgRNA that incorporates a binding site sequence Ms2-binding-sequence for the Ms2 protein. Concurrently, the disclosure fuses the Ms2 protein with Lans5, a modified version of a photosensitive element Lans4, to obtain a fusion protein Ms2-Lans5. Upon illumination of light with a specific wavelength, the fusion protein Ms2-Lans5 undergoes structural changes, enabling the fusion protein Ms2-Lans5 to bind to the Ms2-binding-sequence on the sgRNA, thereby altering the intracellular localization of the sgRNA. By this approach, the disclosure can precisely control distribution and localization of the sgRNA within cells, thereby achieving regulation of a gene editing process. To achieve this objective, the disclosure explores various construction methods and ultimately develops this novel strategy, which provides a new perspective for gene editing technologies.

To sum up, the disclosure makes adjustments to the entire system: two modules initially used in experiments, namely a control element and the sgRNA element, are optimized to be split into three modules after multiple rounds of experiments, thereby ensuring that the size of the fusion proteins does not affect binding between the dCas9 and the sgRNA.

For this purpose, the disclosure adopts a strategy of the Ms2 protein binding to the sgRNA with the Ms2-binding-sequence, which separates the light-controlled element from the gene editing element, allowing a plasmid to enter the cell nucleus more effectively.

After the above series of explorations and screenings, a light-controlled CRISPR component of the disclosure has been finally determined, forming an effective light-controlled gene regulation system (including the light-controlled element NES-Ms2-Lans5, an sgRNA-modified gene targeting element, and a gene regulatory element dCas9-hsf1-vpr). These elements exhibit excellent performance in an optimized plasmid system, significantly improving success rates and effectiveness of the experiments. This provides a more efficient approach for constructing light-controlled gene expression plasmids and lays a solid foundation for subsequent experimental studies.

In some embodiments of the disclosure, by combining a CRISPR-dCas9 system, the RNA-binding protein Ms2, and the photosensitive protein Lans5, a light-controlled gene regulation method has been developed that enables activation of specific genes via illumination. This method primarily consists of three key components: the gene regulatory element, the light-controlled element, and the gene targeting element.
1. The gene regulatory element such as dCas9-hsf1-vpr: this element has a function of activating genes and is internally composed of NLS, dCas9, hsf1, and vpr. The NLS is used to guide fusion proteins to localize within the cell nucleus, a dCas9 protein is used to bind to specific genes, and hsf1-vpr is responsible for activating gene expression.
2. The light-controlled element such as NES-Ms2-Lans5: this element is responsible for converting light signals into cell localization signals. The Ms2 protein can bind to the sgRNA-modified gene targeting element with the Ms2-binding-sequence. The NES ensures that the fusion protein can be transported out of the cell nucleus in an absence of light, while Lans5 is a photosensitive element obtained by deleting an N-terminal NES sequence from Lans4 with a Lov2 structure. The Lans5 can undergo conformational changes under illumination of blue light and bring the fusion protein into the cell nucleus through its NLS. This light-controlled element is located in cytoplasm under dark conditions and enters the cell nucleus under illumination.
3. The sgRNA-modified gene targeting element: this element provides an ability to target specific genes and consists of two main parts: sgRNA and Ms2-binding-sequence. The sgRNA achieves precise localization to a specific gene by forming complementarity with a sequence of a target gene. Meanwhile, the Ms2-binding-sequence specifically binds to the Ms2 protein of the light-controlled element.

When these three elements are transfected into target cells, under conditions without illumination, the gene regulatory element resides in the cell nucleus but does not bind to DNA. Meanwhile, the light-controlled element and the gene targeting element, upon binding to each other to form a first complex, are located in the cytoplasm and do not activate gene expression. When the target cells are exposed to blue light, the Lans5 photosensitive protein in the light-controlled element responds to a light signal, guiding the first complex formed by the light-controlled element and the gene targeting element into the cell nucleus to form a second complex with the gene regulatory element, thereby achieving activation and expression of specific target genes.

To achieve objectives, the disclosure uses the following technical solutions.
1), a photosensitive element Lans4 plasmid is synthesized, and light-control effect of the photosensitive element Lans4 plasmid is verified through cell transfection experiments.
2), the light-controlled element NES-Ms2-Lans5 is constructed, screened, and verified, and the gene regulatory element is optimized to dCas9-hsf1-vpr.
3), a vector for the sgRNA-modified gene targeting element is constructed, and an sgRNA-modified specific to the target gene GAPDH and an sgRNA-modified specific to the target gene NKX2-5 are constructed.
4), illumination activation tests are performed on the human GAPDH and NKX2-5 genes separately.

Base composition of the light-controlled element NES-MS2-Lans5-mCherry and its enzyme cleavage sites at both ends is as follows (GSGSGSG-linker, as shown in SEQ ID NO: 15) CCTAGG (mCherry gene fragment) ACTAGTTAA.

Specifically, sequence composition of the mCherry gene fragment is as shown in SEQ ID NO: 4. Amino acid sequence composition of the light-controlled element NES-MS2-Lans5 is as shown in SEQ ID NO: 6.

An hsf1 gene fragment is as shown in SEQ ID NO: 7. Key base composition of the sgRNA-modified gene targeting element is as shown in SEQ ID NO: 8, where underlined parts are BsmBI recognition sites.

### Embodiment 1

### Lans4-mCherry is Synthesized, and Function and Intracellular Localization Effect of the Photosensitive Element Lans4 Plasmid are Verified.

1.1 Tsingke Biotechnology Co., Ltd. is entrusted to synthesize a Lans4-mCherry plasmid, using cytomegalovirus DNA 3.1 (pcDNA3.1) plasmid as a vector and Lans4-mCherry as an insertion fragment. Specifically, the mCherry gene fragment is a red fluorescent protein used for laboratory signal detection. A final light-controlled element is resynthesized to omit the mCherry gene fragment.

A conventional preparation method for the Lans4-mCherry plasmid is as follows. The pcDNA3.1 plasmid is used as the vector; double digestion is performed on the pcDNA3.1 plasmid with NheI and NotI to obtain a linearized pcDNA3.1 vector (5344 base pairs, abbreviated as bp). The Lans4 gene fragment (495 bp, Kozak sequence+Lans4) and mCherry (717 bp, mCherry+termination codon) gene fragment are prepared for homologous recombination, where gene synthesis yields a Lans4-mCherry fragment with homologous arms. During a homologous recombination reaction, a molar ratio of the Lans4-mCherry fragment to the linearized pcDNA3.1 is 3:1, with a total mass of 50 ng and a total volume of 10 μL. The homologous recombination reaction is performed at 50° C. for 30 min (using a Uniclone One Step Seamless Cloning Kit (SC612) from Genesand Biotech Co., Ltd) to obtain a recombination product. The recombination product is rapidly transformed into competent cells, followed by culturing. Single colonies are picked and sent to a company for sequencing identification, thereby obtaining the Lans4-mCherry plasmid.
1.2 The Lans4-mCherry plasmid is transfected into human embryonic kidney (HEK) 293T cells, and blue light illumination is performed after 48 h of transfection.
1.2.1 Cell culture

Day before (20-24 hours prior): approximately 80,000 HEK 293T cells are seeded into each of wells of a 12-well plate. The 12-well plate seeded with the HEK 293T cells is cross-shaken to make the HEK 293T cells in each of the wells of the 12-well plate distribute uniformly. The 12-well plate is then placed in an incubator to make a density of the HEK 293T cells reach 60-80% on a day of transfection.
1.2.2 Replacing fresh culture medium

Culture medium in each of the wells of the 12-well plate is replaced before a transfection step to ensure a final volume of the culture medium in each of the wells of the 12-well plate of approximately 1 milliliter (mL).
1.2.3 Preparation of transfection reagents

Lipofectamine™ 3000 transfection reagent is used for transfection.

Plasmid preparation in tube B: 50 μL of optimized minimal essential medium (Opti-MEM) reduced-serum medium is added to a first sterile centrifuge tube. 1 microgram (μg) of Lans4-mCherry plasmid DNA and 2 μL of P3000 reagent (an enhancer reagent specifically designed to work synergistically with the Lipofectamine™ 3000) are taken and added to the reduced-serum medium. The Lans4-mCherry plasmid DNA, the P3000 reagent, and the reduced-serum medium are gently mixed to obtain a first mixed solution.

Lipofectamine™ 3000 preparation in tube A: 2 μL of the Lipofectamine™ 3000 is added to a second sterile centrifuge tube, and the Lipofectamine™ 3000 in the second sterile centrifuge tube is diluted with 50 μL of the Opti-MEM reduced-serum medium. The Opti-MEM reduced-serum medium and the Lipofectamine™ 3000 are gently mixed to obtain a second mixed solution.
1.2.4 Formation of transfection complex

The Lans4-mCherry plasmid DNA is mixed with the transfection reagent Lipofectamine™ 3000 by adding the first mixed solution in the tube B to the second mixed solution in the tube A, and the first mixed solution and the second mixed solution are gently mixed to obtain a third mixed solution. The third mixed solution is incubated at room temperature for 10 min to 15 min to form a DNA-Lipofectamine™ 3000 complex (i.e., the transfection complex).
1.2.5 Transfection operation

The transfection complex is added by dropwise adding 50 μL of the DNA-Lipofectamine™ 3000 complex to each of the wells of the 12-well plate, and the 12-well plate is gently shaken in an “8” shape to mix the DNA-Lipofectamine™ 3000 complex, the culture medium, and the HEK 293T cells well.
1.2.6 After transfection for 48 h, illumination tests are performed, and specific methods are as follows:
blue light illumination: irradiating the HEK 293T cells in the 12-well plate transfected with the Lans4-mCherry plasmid using a blue light with a wavelength of 460 nm to 470 nm for 10 min;
stopping illumination: stopping the illumination for 40 min and recording status of the HEK 293T cells; and
confocal microscopy observation: observing changes in distribution of red fluorescent signals of mCherry in the cell nucleus and cytoplasm under a confocal microscope, and recording fluorescence distribution.
1.3 Results analysis

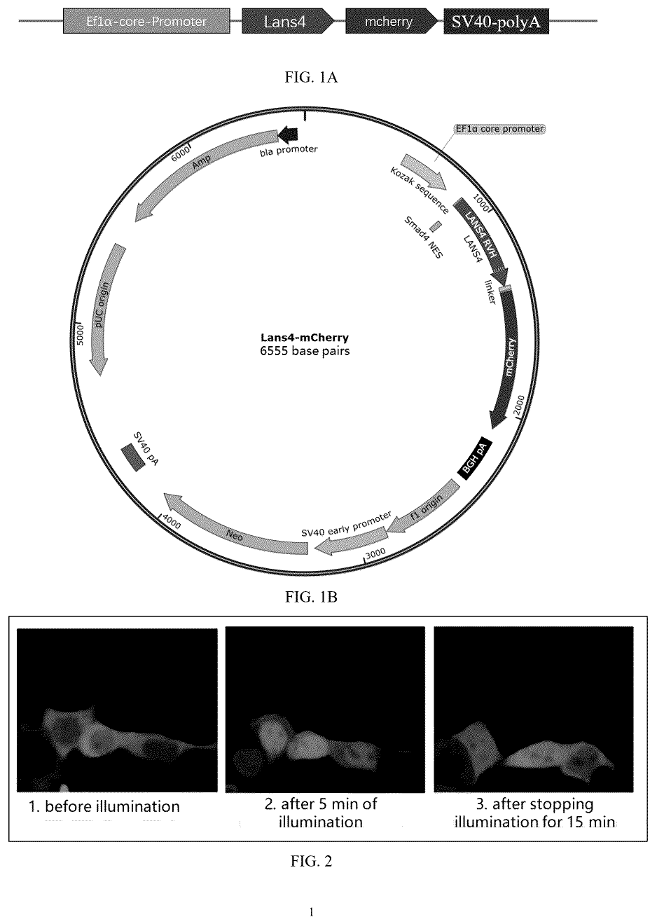

Schematic composition diagrams of the constructed Lans4-mCherry plasmid are shown in FIG. 1A through FIG. 1B.

As illustrated in FIG. 2, it can be seen from the illumination tests that, during a change process from 0 min to 5 min, under the absence of light (silent state), the Lans4-mCherry plasmid is primarily distributed in the cytoplasm, with a nuclear region initially appearing dark or lacking fluorescence. As an illumination duration increases, the nuclear region gradually becomes filled with the red fluorescent signals of the mCherry, indicating that the Lans4-mCherry plasmid enters the cell nucleus under light control. After stopping the illumination, the Lans4-mCherry plasmid follows a reverse process of illumination, returning to its original dark or non-fluorescent state, indicating that the Lans4-mCherry plasmid moves back to the cytoplasm after stopping the illumination. Verification results demonstrate that the Lans4-mCherry has good performance.

### Embodiment 2

The Light-Controlled Element NES-Ms2-Lans5 is Constructed, Screened, and Validated, and the Gene Regulatory Element is Optimized to dCas9-Hsf1-Vpr.
2.1 The pcDNA3.1 plasmid is used as the vector; double digestion is performed on the pcDNA3.1 plasmid with the NheI and the NotI to obtain the linearized pcDNA3.1 vector (5344 bp). An Ms2-Lans4-mCherry gene fragment (1620 bp) is prepared for homologous recombination, where gene synthesis yields an Ms2-Lans4-mCherry fragment with homologous arms. During a homologous recombination reaction, a molar ratio of the Ms2-Lans4-mCherry fragment to the linearized pcDNA3.1 vector is 3:1, with a total mass of 50 ng and a total volume of 10 μL. The homologous recombination reaction is performed at 50° C. for 30 min (using the Uniclone One Step Seamless Cloning Kit (SC612) from Genesand Biotech Co., Ltd) to obtain a recombination product. The recombination product is rapidly transformed into competent cells, followed by culturing at 37° C. for 12 h to 16 h. Single colonies are picked and sent to a company for sequencing identification, thereby obtaining a Ms2-Lans4-mCherry plasmid.

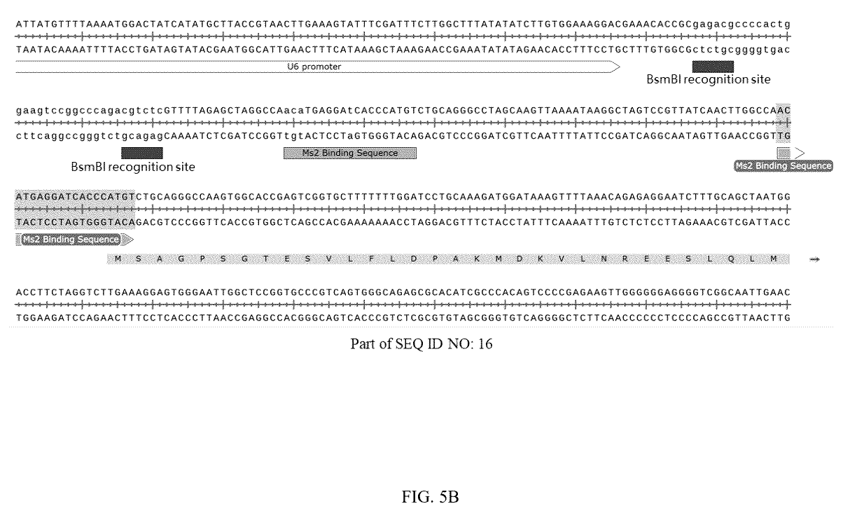

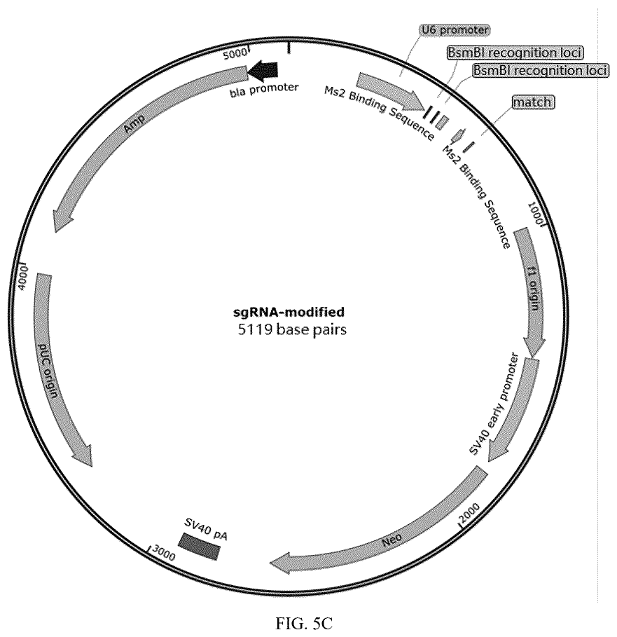

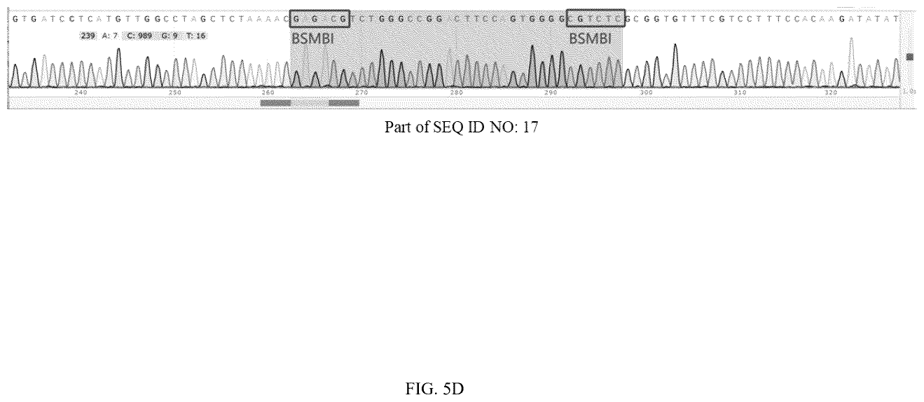

After repeating the above steps and undergoing multiple element position adjustments and modifications (an N-terminal NES sequence is omitted from photosensitive protein Lans4 to obtain Lans5), four versions of plasmids, including N-1 to N-4, are constructed:
N-1: Ms2-Lans4-mCherry;
N-2: Lans4-Ms2-mCherry;
N-3: NES-Ms2-Lans4-Cherry;
N-4: NES-Ms2-Lans5-Cherry (Lans 5 is obtained by omitting the N-terminal NES sequence from Lans4).
2.2 Illumination effect test: transfection conditions are the same as step 1.2 in embodiment 1, and plasmids N-1 to N-4 are tested separately.
2.3 Construction of the plasmid dCas9-hsf1-vpr: a dCas9-vpr plasmid is purchased from Miaoling Biology. The dCas9-vpr plasmid already contains the NLS, dCas9, and a vpr activation domain. However, actual testing reveals that using only the dCas9-VPR plasmid cannot effectively activate expression of a target gene (as illustrated in FIG. 5E), resulting in poor activation efficiency.

The disclosure designs and synthesizes a DNA fragment containing the hsf1 gene (SEQ ID NO: 7) and specifically inserts the DNA fragment containing the hsf1 gene into the dCas9-vpr plasmid through molecular cloning technology to construct the dCas9-hsf1-vpr plasmid. The dCas9-hsf1-vpr plasmid additionally integrates heat shock response enhancing effect of the hsf1 gene to further improve the activation efficiency.
2.4 Construction steps: the dCas9-vpr plasmid is linearized using single-enzyme digestion with MluI to obtain a linearized dCas9-vpr vector. Tsingke Biotechnology Co., Ltd. is entrusted to construct the hsf1 gene with homologous arms. A homologous recombination reaction is performed on the hsf1 gene and the linearized dCas9-vpr plasmid at 50° C. for 30 min with a molar ratio of hsf1 (SEQ ID NO: 7) to the linearized dCas9-vpr plasmid of 3:1, a total mass of 50 ng, and a total volume of 10 μL (using the Uniclone One Step Seamless Cloning Kit (SC612) from Genesand Biotech Co., Ltd) to obtain a recombination product. The recombination product is rapidly transformed into competent cells, followed by culturing, sequencing, and identifying to thereby obtain the dCas9-hsf1-vpr plasmid.
2.5 Results analysis:

N-1 to N-3 versions of the light-controlled elements can enter the cell nucleus under illumination; however, after stopping the illumination, these elements do not move back to the cytoplasm. In contrast, N-4 version of the light-controlled elements can better achieve a process of entering and exiting the cell nucleus controlled by illumination. Therefore, the N-4 version of the light-controlled elements (NES-Ms2-Lans5-mCherry) is successfully constructed. A final light-controlled element used omits the red fluorescent protein mCherry used for signal detection.

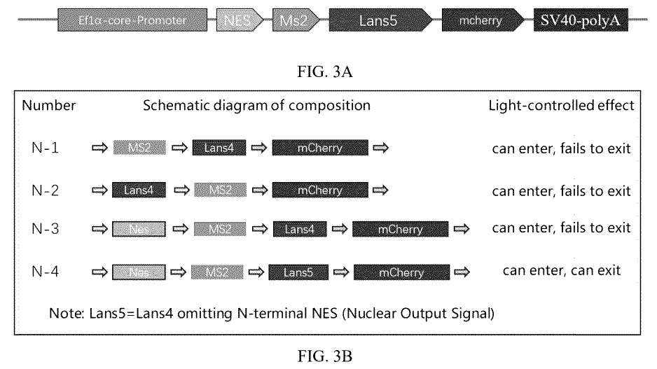

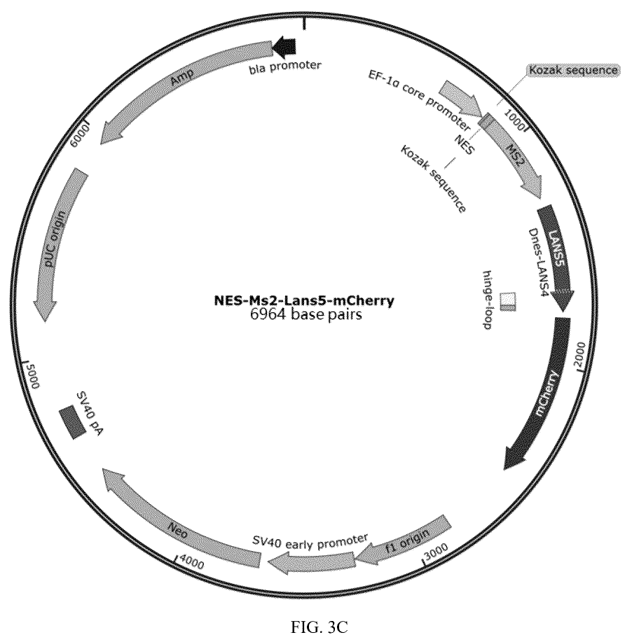

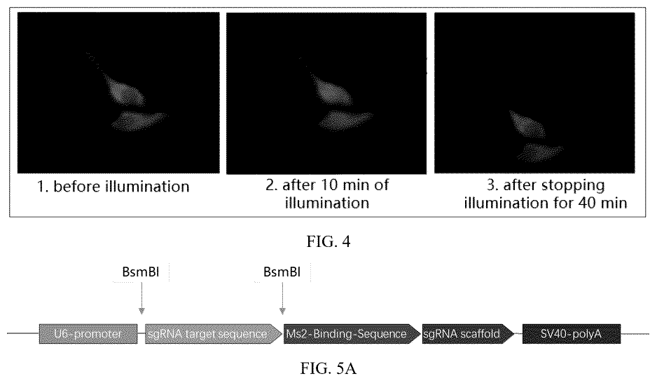

FIG. 3A illustrates a schematic diagram of a linear vector of the light-controlled element NES-Ms2-Lans5-mCherry, and FIG. 3B illustrates a schematic diagram of linearized vectors and light-controlled results for four versions of plasmids (N-1 to N-4). Specifically, the N-1 to N-3 versions of the light-controlled elements can enter the cell nucleus under the illumination but fail to exit the cell nucleus after stopping the illumination. The N-4 version of the light-controlled elements can enter the cell nucleus under the illumination and smoothly exit the cell nucleus after stopping the illumination, which meets experimental requirements. FIG. 3C illustrates a plasmid map of the light-controlled element NES-Ms2-Lans5-mCherry. FIG. 4 illustrates performance of the NES-Ms2-Lans5-mCherry plasmid under the illumination. During a 10 min illumination process, it can be seen that the red fluorescent signals gradually accumulate toward the cell nucleus (indicated by black circles in the figure). At 10 min, the cell nucleus is completely replaced by the red fluorescent signals, demonstrating that the NES-Ms2-Lans5-mCherry plasmid successfully enters the cell nucleus. Within 40 minutes after stopping the illumination, a reverse process of the NES-Ms2-Lans5-mCherry plasmid exiting the cell nucleus is observed, indicating that a blue-light control effect of this light-controlled element is good.

### Embodiment 3

Constructing the Vector for the sgRNA-Modified Gene Targeting Element, the sgRNA-Modified Specific to the Target Gene GAPDH, and the sgRNA-Modified Specific to the Target Gene NKX2-5
3.1 Construction of the vector for the sgRNA-modified gene targeting element: the pcDNA3.1 plasmid is used as the vector, a gene sequence corresponding to an sgRNA with an Ms2-binding-sequence is synthesized by Tsingke Biotechnology Co., Ltd. Simultaneously, two BsmBI restriction sites are introduced at a front end of an inserted sequence. Recognition sites of the two BsmBI restriction sites are 5′-CGTCTC(N)1↓-3′ and 3′-GCAGAG(N)5↑-5′. A unique recognition pattern of BsmBI is utilized to facilitate rapid construction of sgRNAs targeting human GAPDH and NKX2-5 genes in subsequent experiments.

Construction process: double enzyme digestion is performed on the pcDNA3.1 plasmid and an Ms2-sgRNA gene synthesis fragment with the MluI and the NheI to obtain the linearized pcDNA3.1 vector and a linearized Ms2-sgRNA gene synthesis fragment. The linearized pcDNA3.1 vector and the linearized Ms2-sgRNA gene synthesis fragment are then linked to each other at room temperature for 1 h, followed by transforming and culturing for 12 h to 16 h. Single colonies are picked and sent to a company for sequencing to obtain the vector for the sgRNA-modified.
3.1 Construction of the sgRNA-modified specific to the target gene GAPDH and the sgRNA-modified specific to the target gene NKX2-5: a target sequence of sgRNA for the target gene GAPDH is designed using an online tool Cas-Designer, obtained by transfecting and screening, and validated to obtain a final target sequence: GCGGGCTCAATTTATAGAAAC (SEQ ID NO: 9). Similarly, a final target sequence for the target gene NKX2-5 is determined as CAGGTAGCGCTGAGCACAAG (SEQ ID NO: 10). Both sequences are constructed into vectors of the sgRNA-modified gene targeting element.
3.1.1 The vector for the sgRNA-modified gene targeting element is linearized by digestion with BsmBI to obtain a linearized sgRNA-modified fragment.
3.1.2 Oligo primers (SEQ ID NO: 9 and SEQ ID NO: 10) for the target gene GAPDH and the target gene NKX2-5 are synthesized by Tsingke Biotechnology Co., Ltd. Sense strands and antisense strands of these primers are mixed in equal proportions and annealed to form a double-stranded oligo primer for the target gene GAPDH and a double-stranded oligo primer for the target gene NKX2-5.
3.1.3 The linearized sgRNA-modified fragment undergoes a ligation reaction with the double-stranded oligo primer for the target gene GAPDH and the double-stranded oligo primer for the target gene NKX2-5, respectively, followed by transformation and growth. After 12-16 hours, single colonies are picked for sequencing.

Results analysis: based on an expected sequence, a correct sgRNA-modified vector is obtained, as illustrated in FIG. 5, and the sgRNA-modified vector is used for inserting target sequences of the target gene GAPDH and the target gene NKX2-5. FIG. 5A illustrates a schematic structure diagram of a plasmid vector, driven by a U6 promoter (a commonly used RNA polymerase III (Pol III)-dependent promoter); FIG. 5B and FIG. 5C illustrate the expected sequence and an expected complete plasmid map, respectively; FIG. 5D illustrates a final sequencing result, which is consistent with the expected sequence, confirming successful construction of the sgRNA-modified.

### Embodiment 4

### Performing Illumination Activation Tests on the Human GAPDH and NKX2-5 Genes Separately

4.1 Co-transfecting the NES-Ms2-Lans5 and the dCas9-hsf1-vpr obtained in embodiment 2 and transfecting the sgRNA-modified (GAPDH or NKX2-5) to verify activation effect
4.1.1 The HEK 293T cells are prepared in the 12-well plate 24 h in advance, ensuring that the density of the HEK 293T cells is 60-80% at the time of transfection. The NES-Ms2-Lans5 and the dCas9-hsf1-vpr are co-transfected.
4.1.2 After the NES-Ms2-Lans5 and the dCas9-hsf1-vpr are co-transfected for 24 h, the sgRNA-modified is transfected (GAPDH or NKX2-5).
4.1.3 72 h after an initial transfection, an illumination group is irradiated with the blue light with a wavelength of 460 nm to 470 nm for 6 h.
4.1.4 After the illumination is completed, RNA is extracted, and during reverse transcription, a real-time fluorescence quantitative polymerase chain reaction test is performed to detect activation effects on the human GAPDH and NKX2-5 genes.
4.2 Results analysis: each target gene is divided into 4 groups of light-controlled CRISPR components: control group (nc): NES-Ms2-Lans5 plasmid, dCas9-hsf1-vpr plasmid, and sgRNA-modified; mock group: no transfection treatment; non-illumination group: co-transfection of three plasmids (NES-Ms2-Lans5 plasmid, dCas9-hsf1-vpr plasmid, and sgRNA-modified plasmid); and illumination group (illumination for 6 h): co-transfection of three plasmids (NES-Ms2-Lans5 plasmid, dCas9-hsf1-vpr plasmid, and sgRNA-modified plasmid).

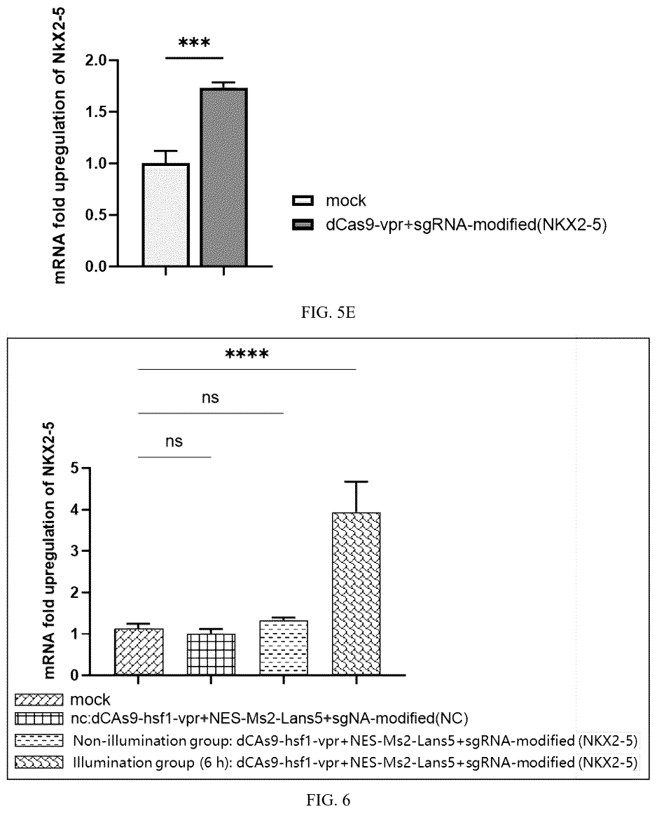

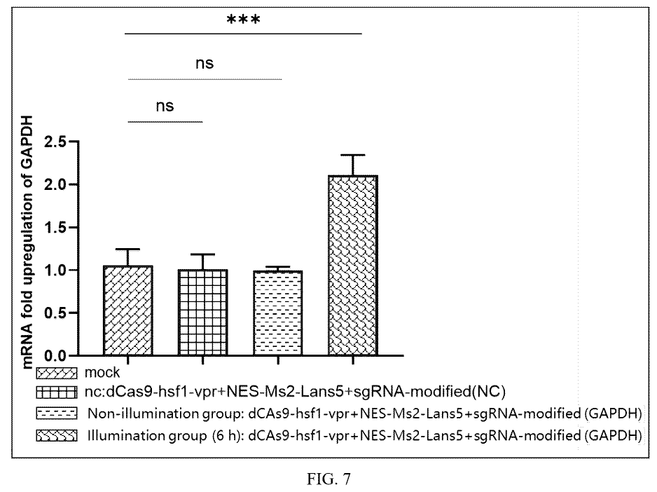

As illustrated in FIG. 6 and FIG. 7, composition of the light-controlled CRISPR component corresponding to the non-illumination group is gene regulatory element+light-controlled element+gene targeting element. Composition of the light-controlled CRISPR component corresponding to the illumination group (6 h) is gene regulatory element+light-controlled element+gene targeting element (GAPDH or NKX2-5).

Non-illumination group: a messenger RNA (mRNA) fold upregulation of the human NKX2-5 gene is similar to that of the nc group, showing an insignificant change.

Illumination group (6 h): an mRNA fold upregulation of the human NKX2-5 gene is significantly increased, reaching approximately 4-fold, and shows a significant difference compared to both the nc group and the mock group.

Experimental results indicate that illumination treatment significantly promotes expression of NKX2-5 mRNA, while the nc group and non-illumination treatment have no significant effect on the expression of NKX2-5 mRNA. The nc group demonstrates that the method designed in the disclosure has strong specificity and high targeting ability, and establishment of the mock group confirms that there are no significant problems with the experimental methods and reagents.

For the two genes GAPDH and NKX2-5, the light-controlled CRISPR component constructed by the disclosure achieved excellent activation effects on the target genes. This indicates that the light-controlled gene editing construction method provided by the disclosure offers a new means for gene regulation. This construction method has advantages of higher spatiotemporal resolution and flexibility. Multiple sgRNAs are designed to simultaneously target multiple gene loci, and the light-controlled activation of the sgRNAs provides reversibility and dynamic regulation capability for gene expression, offering new tools for the treatment of multigene diseases.

Various technical features of the above embodiments can be combined in any way. In order to simplify description, not all possible combinations of the various technical features in the above embodiments have been described. However, as long as there is no contradiction in combination of these technical features, they should be considered within a scope of this specification.

The above embodiments only express several embodiments of the disclosure, and their descriptions are more specific and detailed, but should not be understood as limiting a scope of protection of the disclosure. It should be pointed out that for those skilled in the art, several modifications and improvements can be made without departing from a concept of the disclosure, and these modifications and improvements fall within the scope of protection of the disclosure. Therefore, the scope of protection of the disclosure should be based on the appended

What is claimed is:

1. A light-controlled element nuclear export signal (NES)-MS2 bacteriophage coat protein (Ms2)-light-activated nuclear shuttling (Lans5), comprising, sequentially in an order from 5′ to 3′, a NES gene fragment, an Ms2 gene fragment, and a Lans5 gene fragment; wherein each gene fragment is linked via a linker;
the Lans5 gene fragment is as shown in SEQ ID NO: 3;
the NES gene fragment is as shown in SEQ ID NO: 1; and
the Ms2 gene fragment is as shown in SEQ ID NO: 2.

2. The light-controlled element NES-Ms2-Lans5 as claimed in claim 1, wherein the linker is the amino acid sequence of GSGSGSGSG as shown in SEQ ID NO: 11 or the amino acid sequence of GGGGSGGGGS as shown in SEQ ID NO: 12.

3. A vector expressing the light-controlled element NES-Ms2-Lans5 as claimed in claim 1.

4. The vector as claimed in claim 3, wherein the vector is a plasmid.

5. A light-controlled clustered regularly interspaced short palindromic repeat (CRISPR) component, comprising the vector as claimed in claim 4, a vector expressing a single guide ribonucleic acid (sgRNA)-modified gene targeting element, and a vector expressing a gene regulatory element dead CRISPR-associated protein 9 (dCas9)-heat shock factor 1 (hsf1)-virus protein r (vpr);
wherein the vector expressing the gene regulatory element dCas9-hsf1-vpr is a plasmid obtained by inserting an hsf1 gene fragment into a dCas9-vpr plasmid; and
wherein the sgRNA-modified gene targeting element comprises an sgRNA target sequence specific to a target gene, an Ms2-binding-sequence capable of specifically binding to the Ms2 gene fragment, and enzyme cleavage sites at both ends of the sgRNA target sequence.

6. The light-controlled CRISPR component as claimed in claim 5, wherein the Ms2-binding-sequence is ACATGAGGATCACCCATGT as shown in SEQ ID NO: 13; and
the sgRNA-modified gene targeting element is the sequence as shown in SEQ ID NO: 8.

7. The light-controlled CRISPR component as claimed in claim 5, wherein the hsf1 gene fragment is as shown in SEQ ID NO: 7.

8. An application method of the light-controlled CRISPR component as claimed in claim 5, comprising:
performing light-controlled gene expression using the light-controlled CRISPR component, wherein the application method is for non-therapeutic purposes.

9. The application method as claimed in claim 8, comprising applying the light-controlled CRISPR component for preparing a kit for the light-controlled gene expression.

10. A method for light-controlled target gene expression for non-therapeutic purposes, comprising the following steps:
S1, acquiring the light-controlled CRISPR component as claimed in claim 5, wherein the light-controlled CRISPR component comprises the vector expressing the light-controlled element NES-Ms2-Lans5, the vector expressing the sgRNA-modified gene targeting element, and the vector expressing the gene regulatory element dCas9-hsf1-vpr;
S2, co-transfecting the vector expressing the light-controlled element NES-Ms2-Lans5 and the vector expressing the gene regulatory element dCas9-hsf1-vpr in cells containing the target gene;
S3, after co-transfecting the vector expressing the light-controlled element NES-Ms2-Lans5 and the vector expressing the gene regulatory element dCas9-hsf1-vpr for 24 hours (h), transfecting the vector expressing the sgRNA-modified gene targeting element; and
S4, performing illumination according to needs of target gene expression; and
wherein a light for the illumination is blue light with a wavelength in a range of 460 nanometers (nm) to 470 nm; and the illumination applies in cycles of 9 minutes (min) to 11 min of light followed by 2.5 min to 3.5 min of dark, for a total duration of 5.5 h to 6.5 h.


SEQUENCE LISTING (17 sequences)

SEQ ID NO: 1 - DNA, 30 bp, synthetic construct
  source 1..30: mol_type = other DNA
ggaattgacctgtctggactgacacttcaa

SEQ ID NO: 2 - DNA, 387 bp, synthetic construct
  source 1..387: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 3 - DNA, 456 bp, synthetic construct
  source 1..456: mol_type = other DNA
ggcagtggaagtggaagccttgccacgacactggagaggatcgagaaaaatttcgttatcaccgacccaaggctgcccgataacccaatcatcttcgcaagcgatagctttctgcagctgaccgaatactcaagggaagagatcctgggccgcaattgtaggtttctgcaggggccagagactgaccgagctacagtgagaaaaatcagagatgccattgataaccagaccgaggtgaccgttcagcttattaattacaccaagtccggtaagaaattttggaacttgtttcacctgcagccaatgcgcgaccagaagggggacgtgcaatatttcatcggagtccagctggatggaactgagagggtacatgacgctgccgaaagagagggtgtcatgcttattaagaaaaccgccgagaacatcgacatggccgccaagcgggtgaagctggat

SEQ ID NO: 4 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 5 - DNA, 960 bp, synthetic construct
  source 1..960: mol_type = other DNA
gccaccatgggaattgacctgtctggactgacacttcaagcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacggatccggcggtggtggttctggtggtggcggttctgatatcggcagtggaagtggaagccttgccacgacactggagaggatcgagaaaaatttcgttatcaccgacccaaggctgcccgataacccaatcatcttcgcaagcgatagctttctgcagctgaccgaatactcaagggaagagatcctgggccgcaattgtaggtttctgcaggggccagagactgaccgagctacagtgagaaaaatcagagatgccattgataaccagaccgaggtgaccgttcagcttattaattacaccaagtccggtaagaaattttggaacttgtttcacctgcagccaatgcgcgaccagaagggggacgtgcaatatttcatcggagtccagctggatggaactgagagggtacatgacgctgccgaaagagagggtgtcatgcttattaagaaaaccgccgagaacatcgacatggccgccaagcgggtgaagctggatgggtctggaagtgggtcggggcctaggactagttaa

SEQ ID NO: 6 - protein, 319 aa, synthetic construct
ATMGIDLSGLTLQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGSGGGGSGGGGSDIGSGSGSLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTERVHDAAEREGVMLIKKTAENIDMAAKRVKLDGSGSGSGPRTS

SEQ ID NO: 7 - DNA, 372 bp, synthetic construct
  source 1..372: mol_type = other DNA
ggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 8 - DNA, 188 bp, synthetic construct
  source 1..188: mol_type = other DNA
gaaacaccgcgagacgccccactggaagtccggcccagacgtctcgttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 9 - DNA, 21 bp, synthetic construct
  source 1..21: mol_type = other DNA
gcgggctcaatttatagaaac

SEQ ID NO: 10 - DNA, 20 bp, synthetic construct
  source 1..20: mol_type = other DNA
caggtagcgctgagcacaag

SEQ ID NO: 11 - protein, 9 aa, synthetic construct
GSGSGSGSG

SEQ ID NO: 12 - protein, 10 aa, synthetic construct
GGGGSGGGGS

SEQ ID NO: 13 - DNA, 19 bp, synthetic construct
  source 1..19: mol_type = other DNA
acatgaggatcacccatgt

SEQ ID NO: 14 - protein, 6 aa, synthetic construct
GSGSGS

SEQ ID NO: 15 - protein, 7 aa, synthetic construct
GSGSGSG

SEQ ID NO: 16 - DNA, 10015 bp, synthetic construct
  source 1..10015: mol_type = other DNA
aattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgagacgtgcacagttaacccccagtggagtcgtctcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatcctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcg

SEQ ID NO: 17 - DNA, 926 bp, synthetic construct
  source 1..926: mol_type = other DNA
gtgatcctcatgttggcctagctctaaaacgagacgtctgggccggacttccagtggggcgtctcgcggtgtttcgtcctttccacaagatatatccggatgcgcttgcactgacgggcaccggagccaattcccactcctttcaagacctagaaggtccattagctgcaaagattcctctctgtttaaaactttatccatctttgcaggatccaaaaaaagcaccgactcggtgccacttggccctgcagacatgggtgatcctcatgttggccaagttgataacggactagccttattttaacttgctaggccctgcagacatgggtgatcctcatgttggcctagctctaaaacgagacgtctgggccggacttccagtggggcgtctcgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgg